(19) United States
(12) Patent Application Publication
(10) Pub. No.: US 2026/0263509 A1
(43) Pub. Date: Sep. 10, 2026

(54) COATED SORBENT MATERIALS FOR THE REMOVAL OF AMMONIUM AND ASSOCIATED METHOD OF USE

(71) Applicant: HEMOCLEANSE TECHNOLOGIES LLC, Lafayette, IN (US)

(72) Inventor: STEPHEN ASH, Lafayette (IN)

(73) Assignee: HEMOCLEANSE TECHNOLOGIES LLC, Lafayette, IN (US)

(21) Appl. No.: 18/875,505

(22) PCT Filed: Jun. 19, 2023
(86) PCT No.: PCT/US23/68683

§ 371 Date: Dec. 16, 2024

Related U.S. Application Data
(60) Provisional application No. 63/353,063, filed on Jun. 17, 2022.

(51) Int. Cl. A61K 33/24; A61K 9/00; A61K 9/50; A61P 3/12; B01D 71/06; B01J 39/12; B01J 41/10

(52) U.S. Cl. CPC A61K 33/24; A61K 9/0053; A61K 9/5015; A61P 3/12; B01D 71/06; B01J 39/12; B01J 41/10

(57) ABSTRACT

An oral sorbent composition and method for use in removing multiple toxins of kidney failure from the gut. The oral sorbent composition having a gas permeable membrane or coating around a sorbent component that would remove small and charged toxins such as sodium, potassium, hydrogen, phosphate and ammonium (from urea) which are plentiful in the gut. The sorbent component can be a hydroxide-loaded zirconium oxide (H—ZP) and coated with a thin gas permeable membrane or coating configured to trap ammonium captured by the oral sorbent composition.

### CROSS-REFERENCE TO RELATED APPLICATIONS

This application claims priority to U.S. Provisional Patent Application No. 63/353,063 filed 17 Jun. 2022, to the above named inventor, and is herein incorporated by reference in its entirety.

### FIELD OF THE INVENTION

The invention relates generally to an oral sorbent mixture having a gas permeable membrane that would remove multiple toxins of kidney failure from the gut. Small and charged toxins such as sodium, potassium, hydrogen, phosphate and ammonium (from urea) are plentiful in the gut. Removing these toxins would diminish serum levels and would help to delay the need for dialysis in many patients, especially the elderly.

### BACKGROUND

Within the prior art there exists extracorporeal blood treatment devices with columns or suspensions of sorbents for removal of toxins in treatment of kidney failure, liver failure, sepsis and SIRS. The sorbents utilized have included carbon, cation exchangers, anion exchangers, and immobilized enzymes. One such sorbent is zirconium cyclo-silicate as an oral sorbent, a cation exchanger that selectively exchanges monovalent cations such as potassium for sodium and hydrogen, but has no binding of divalent cations such as calcium or magnesium. This oral sorbent is now FDA approved to market in treatment of hyperkalemia in patients with renal insufficiency or heart failure (brand name, Lokelma).

A combination of hydrogen-loaded zirconium phosphate, a classic non-selective cation exchanger (H—ZP) with hydroxide-loaded zirconium oxide, a non-selective anion exchanger (OH—ZO) has shown effectiveness in binding toxins. The combination binds cations and anions simultaneously, releasing H+ and OH−, which combine to make water. From in vitro studies using fluids simulating the contents of the small bowel and colon, the ZP/ZO combination is very effective in removing all of the sodium, potassium, hydrogen, and phosphate with the exception of ammonium (from urea). The binding of ammonium is less than 1 mEq/gram of ZP, which is insufficient given the huge amounts of urea in the body water and in daily production. Other cation and anion exchangers have similar affinities. The reason for choice of the inorganic zirconium cation and anion exchangers for the sorbent therapy is that they are more palatable than resins, and these compounds have been used for years in regeneration of dialysate, where they've been proven to be free of contaminants and safe to use in patients with kidney failure. Therefore, there is a need to create an oral sorbent using a H—ZP that selectively removes ammonium (NH₃) from solution. The goal is to have a binding of 7-10 mEq of ammonium per gram of zirconium phosphate particles (ZP).

### BRIEF SUMMARY OF THE INVENTION

In one aspect, this disclosure is related to an oral sorbent composition for ammonium removal. The composition can include cation exchange materials for sorbent material that can be coated with or encapsulated by a gas-permeable and/or hydrophobic membrane. The sorbent materials can utilize a hydrogen loaded sorbent material, including but not limited to zirconium phosphate (ZP) or citric acid.

In another aspect, this disclosure is related to a method of removing ammonium from animals. The method can include providing an oral sorbent composition comprising at least one cation exchange material that is coated or encapsulated by a gas permeable membrane. The gas permeable membrane can allow gas to pass through the membrane but not liquid water. In some embodiments, the membrane can allow for the passage of water vapers through the membrane at low pH level.

### BRIEF DESCRIPTION OF THE DRAWINGS

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

FIG. 1 provides a diagram illustrating an acid trap created by using a small fraction of NH₃ which co-exists with NH₄⁺ in solution, wherein the NH₃ diffuses across the gas-permeable membrane to combine with the H⁺ on the ZP and form NH₄⁺; the NH₄⁺ is thus “trapped” inside the membrane, according to the present disclosure.

FIG. 2A is a chart illustrating that flat sheet gas-permeable membranes were found effective at slowly transferring NH₃ to a suspension of H—ZP, according to the present disclosure.

FIG. 2B is a chart illustrating the pH of solutions and sorbents during NH₃ transfer through the gas-permeable membrane.

FIG. 3 is a chart illustrating that NH₄⁺ transfer is slow and H—ZP uses only a small portion of its total capacity (approximately 8 meq cation bound per gram of ZP) during the period of binding due to the relatively small area of the gas-permeable membrane.

FIG. 4A are electron microscope images of the appearance of the coated H—ZP, according to the present disclosure.

FIG. 4B are enhanced images of the appearance of the coated H—ZP, according to the present disclosure showing the uncoated versus PDMS coated ZP absorption.

FIG. 5 is a chart illustrating the permeability of the membrane-coated H—ZP to ammonium of the present disclosure, and it was bound at a slow rate, according to the present disclosure.

FIG. 6 is a chart illustrating the relative mass transfer coefficients at 3 hours.

FIG. 7 is a chart illustrating the relative mass transfer coefficients at 24 hours.

FIG. 8 is a chart illustrating the change of the mass transfer coefficient during an experiment.

FIG. 9 is a chart illustrating the change of the mass transfer coefficient during an experiment.

FIG. 10 is a chart illustrating the amount of total nitrogen adsorbed by the sorbent at 24 hours as a fraction of the total available hydrogen.

FIG. 11 is a chart illustrating the actual mass transfer coefficients.

FIG. 12 is a chart illustrating the actual mass transfer coefficients.

FIG. 13 is an illustration of an uncoated zirconium phosphate particle of the present disclosure and a zirconium phosphate particle coated with a gas permeable membrane.

### DETAILED DESCRIPTION OF THE INVENTION

The following detailed description includes references to the accompanying drawings, tables and charts which all form a part of the detailed description. The drawings, charts, and tables, show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention of this disclosure is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit, or scope of the present invention. All such modifications are intended to be within the scope of the disclosure made herein.

Unless otherwise indicated, the words and phrases presented in this document have their ordinary meanings to one of skill in the art. Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer to embodiments of the invention that may afford certain benefits, under certain circumstances. However, other embodiments may also be preferred, under the same or other circumstances.

Furthermore, the recitation of one or more preferred embodiments does not imply that other embodiments are not useful and is not intended to exclude other embodiments from the scope of the invention.

As used herein, the terms “front,” “back,” “rear,” “upper,” “lower,” “right,” and “left” in this description are merely used to identify the various elements as they are oriented in the FIGS, with “front,” “back,” and “rear” being relative to the apparatus. These terms are not meant to limit the elements that they describe, as the various elements may be oriented differently in various applications.

As used herein, the term “coupled” means the joining of two members directly or indirectly to one another. Such joining may be stationary in nature or movable in nature. Such joining may be achieved with the two members, or the two members and any additional intermediate members being integrally formed as a single unitary body with one another or with the two members or the two members and any additional intermediate members being attached to one another. Such joining may be permanent in nature or alternatively may be removable or releasable in nature. Similarly, coupled can refer to a two member or elements being in communicatively coupled, wherein the two elements may be electronically, through various means, such as a metallic wire, wireless network, optical fiber, or other medium and methods.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure.

Following are more detailed descriptions of various concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

The disclosed invention of the present disclosure most generally relates to an oral composition that can include providing a gas-permeable membrane and/or hydrophobic coating on and around an ammonium sorbent component, for the purpose of removing ammonium. The oral composition of the present disclosure can be very useful for patients that have renal diseases such as end-stage renal disease (ESRD). More generally the disclosure provides a gas-permeable membrane/coating on any type of sorbent component for ammonia or ammonium, whether organic or inorganic. The membrane or coating can be applied to the sorbent material in one or more layers. The sorbent component material can be an ion exchange material. In some exemplary embodiments, the sorbent material can be hydrogen-loaded cation exchange material.

Accordingly, the disclosure also relates to any type of gaseous toxins in solution that can be trapped within an encapsulated sorbent by binding, for example CO₂. CO₂ dissolved in blood exists as carbonic acid, which dissociates to CO₂ and H₂O. CO₂ crossing a membrane can bind with a sorbent such as Ca(OH)₂, forming insoluble CaCO₃. A gas permeable membrane and/or hydrophobic coating covering Ca(OH)₂ would prevent small amounts of Ca⁺⁺ and OH⁻ which dissolve from Ca(OH)₂ from passing to the blood or surrounding fluid (similar to how a gas permeable membrane can prevent NH₄⁺ from passing to a solution on the other side of the membrane). In some exemplary embodiments, the membrane can allow gas through but not liquid water. The membrane can similarly allow for water vapor transfer for low pH.

In some exemplary embodiments, the sorbent material can be a zirconium phosphate. Additionally, the zirconium phosphate can be hydrogen loaded to be further promote capture of NH₄⁺ generated by urease action on urea. The sorbent material can then be coated or encapsulated by a membrane or coating. In one exemplary embodiment, the coating can be a gas-permeable membrane to increase binding capacity for NH₄⁺ and selectivity in the presence of other ions. The coating can provided a barrier to ions in the patients digestive system, while allow for the gaseous ammonia to be transferred to the sorbent composition inside the coating and bind with H⁺ to form NH₄⁺, and thus be trapped within the capsule. The use of the ZP as the sorbent also reduced or even eliminated osmotic pressure of the oral composition.

The present disclosure also provides a method of removing NH₄⁺ withing a patient's intestines or gut to reduce blood urea levels and diminish the need for blood dialysis treatments. An oral composition can include an oral sorbent component can be coated with and/or encapsulated by a gas-permeable membrane coating prior to be ingested by the patient. The oral composition can then be provided and ingested by the patient. Administering one or more pharmaceutical formulation prepared according to the present invention serve to reduce the frequency of dialysis treatments. Additionally and possible more important from a patient's standpoint, present invention can allow a patient to ingest a more “normal diet”—other than taking one or more of the pharmaceutical preparations—and still significantly reduce the patient's toxin levels. The pharmaceutical preparation can be specifically formulated to correspond the patient' diet. This can facilitate better patient compliance with required treatment/medications and contribute to the patient's overall mental state and physical health.

The coating and or membrane can be any suitable material to provide for selective binding to ammonium and no binding to other ions. In some exemplary embodiments, the gas permeable coating can be applied using a thermal deposition technique or procedure. Various membranes or coating can be applied including but not limited to one or more polymers, polyethylene, polyimide, polysiloxane, hydrophobic polydimethylsiloxane (PDMS) or a perfluorooctyltriethoxysilane (FOTS) coating. In some exemplary embodiments an intermediary coating can be applied to better form the gas-permeable membrane coating. An intermediary coating can include tetraethyl orthosilicate. The gas permeable membrane coating will not interfere the ability of the sorbent material's absorption capacity.

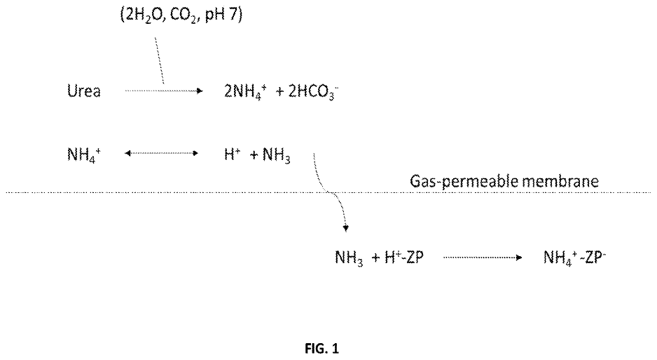

Referring now generally to FIGS. 1-5 and embedded tables and charts, to provide an oral sorbent composition. In some exemplary embodiments, the oral sorbent composition will have a selectivity for ammonium (NH₃) from solution and can include a gas-permeable membrane coating for sorbent materials within the composition. The sorbent materials of the composition can include any suitable materials having selectivity for ammonium from solution.

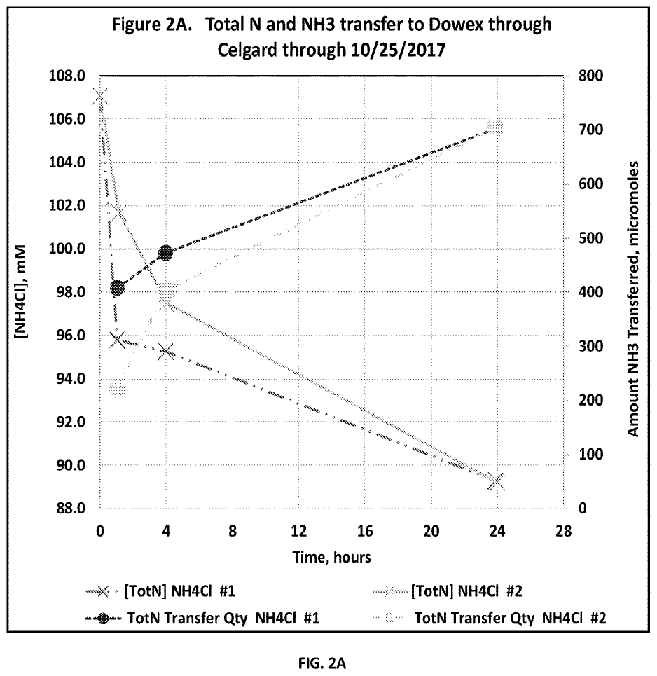

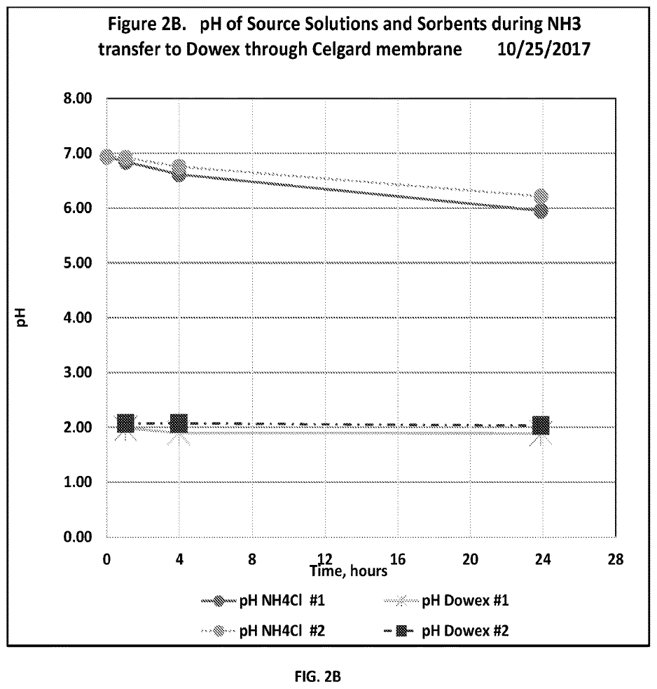

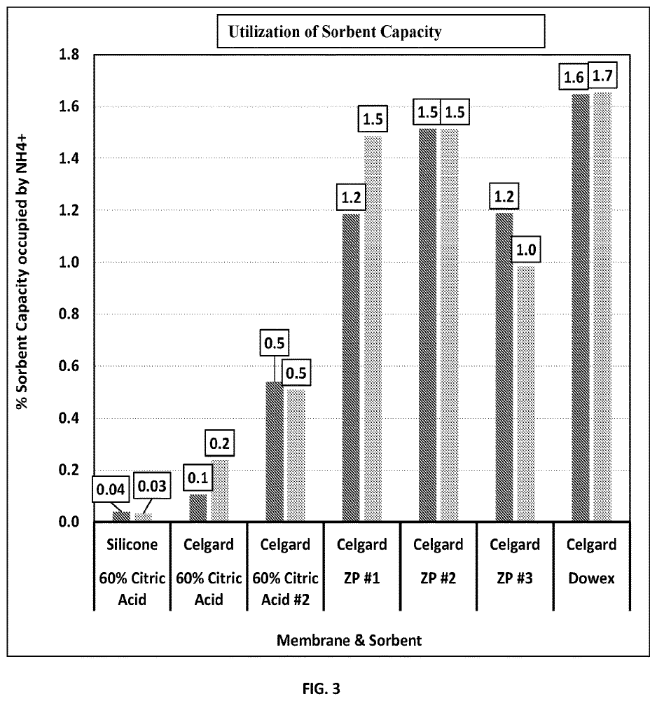

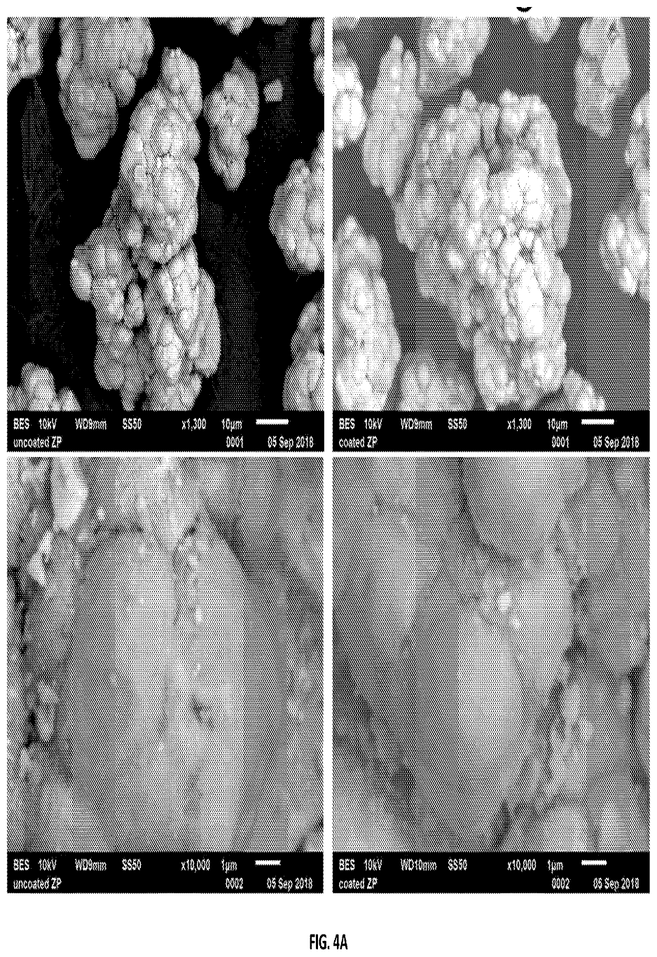

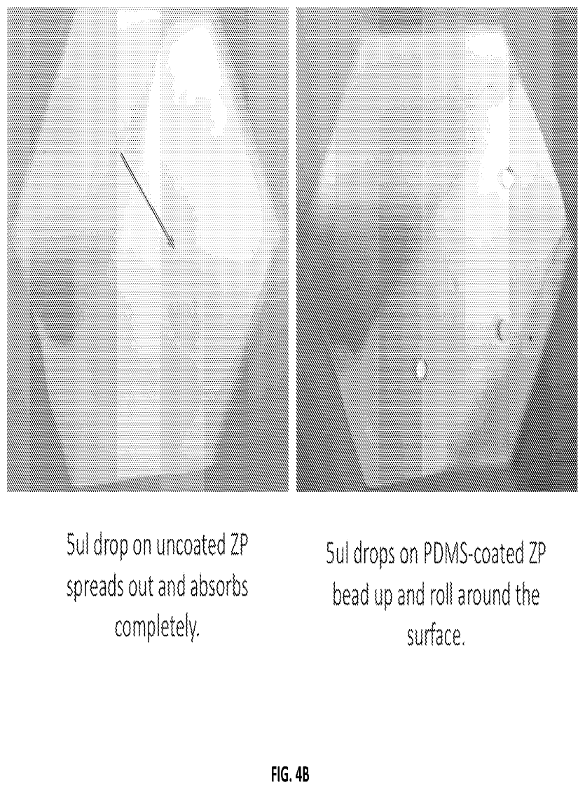

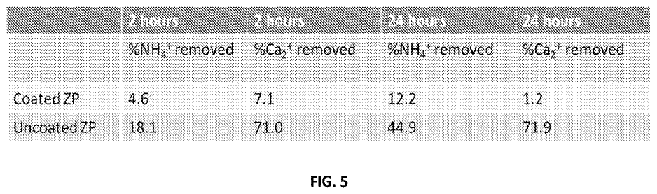

In some exemplary embodiments, the sorbent materials can include H—ZP is utilized. Even at neutral pH, there is a small fraction of NH₃ which co-exists with NH₄⁺ in solution; the NH₃ diffuses across the gas-permeable membrane to combine with the H⁺ on the ZP and form NH₄⁺. The NH₄⁺ is thus “trapped” inside the membrane as shown in FIG. 1. This reaction can take place with no osmotic force being generated. NH₃ is a small fraction of NH₄⁺ but can be continually replenished when it is removed from solution. To test the concept, initial experiments with flat sheet gas-permeable membranes were utilized and found effective at slowly transferring NH₄ (as NH₃) to a suspension of H—ZP as shown in FIGS. 2A-B. The resulting capacity of the sorbent is large. Chemical testing showed that the resulting capsules were impermeant to calcium and magnesium but did allow passage of some ammonium, at a slow rate as shown in FIG. 3. The total amount of NH₄⁺ bound by the H—ZP in 24 hours was significantly less than its maximal capacity for cation binding (about 8 meq/gram). Preliminary studies with thermal coating of H—ZP with a gas permeable membrane with siloxane (PDMS) were performed in 2019. Thermal deposition of the PDMS was the technique used. The appearance of the coated H—ZP is shown in FIGS. 4A-B. FIG. 5 shows the removal of NH₄⁺ and Ca⁺⁺ from a solution by PDMS-coated H—ZP. There was slow but progressive binding of NH₄⁺ by the coated H—ZP, over a 24 hour period. There was almost no binding of Ca⁺⁺ during this time period. By comparison, uncoated H—ZP bound much more Ca⁺⁺ than NH₄⁺. These tests were in NH₄⁺ solutions which had a low pH. It is anticipated that the transfer of NH₃ would have been much faster at a neutral pH. In the “best case” of binding of 5.5 mEq/gram of NH₄⁺, 65 grams of coated H—ZP could bind 100% of daily urea production in a patient, within the gut, depending on dietary intake of protein

### Experimental Section

To establish the feasibility of using various sorbent materials to remove ammonium from aqueous solutions, several experiments were undertaken. Within these experiments the sorbent is separated from the solution by a hydrophobic membrane in order to increase the specificity of ammonium sorption.

Ultimately, an ammonia sorbent will be a component of an orally administered sorbent, to be ingested with food, that would balance electrolytes and remove unwanted metabolites from kidney failure patients.

It is desirable to remove a large quantity of nitrogen (5 grams/day) from ESRD patients. Currently, no known specific sorbents for ammonia or ammonium exist, so an attempt was made to make the effect of non-specific sorbents specific for absorption of nitrogenous compounds by membrane separation. Ammonium ion is in equilibrium with small concentrations of ammonia gas in the small bowel contents. A hydrophobic membrane can allow for the passage of gaseous ammonia while excluding all dissolved electrolytes. The ammonia can then be sequestered in the sorbent compartment and eliminated with the stool.

Membranes and test concentrations were chosen for high transfer rates so that the transfer could be easily measured. Sorbent capacity was greatly in excess of the ammonia transfer. In the ammonium chloride source solution, ammonium and ammonia are in an equilibrium which is determined by the solution pH and shown below as Equation 1.

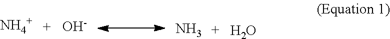

Equilibrium can be formed in ammonia compounds in aqueous solution. However, the NH₃ will diffuse to the sorbent solution, where the [NH₃] is much lower, resulting in a net continuous production of ammonia as illustrated in Equation 2.

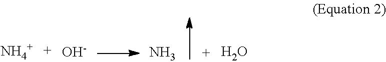

Loss of ammonia to sorbent can then produce a net increase of [H⁺] in solution. Neutralization of OH⁻ by this reaction will result in decreased pH of ammonium chloride solution. After NH₃ diffuses into the sorbent solution, it can experience the same equilibrium reactions as Equation 1. In the presence of a hydrogen-loaded sorbent, either a cation exchanger such as ZP or citric acid, the reaction sequence will be either Equation 3A or 3B.

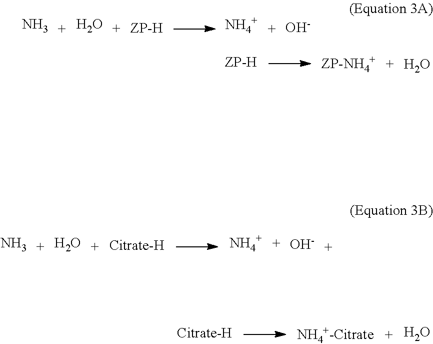

Ammonia bound to sorbent, solution pH unchanged. The reaction is driven towards adsorption primarily by the concentrations of the reactants. ZP has highest affinity for H⁺ but that is overcome by other factors influencing the reaction. Note that the pH of the sorbent solution should not change if this is the only method for binding ammonium.

Experimental membranes were selected from materials readily available in the lab. Three such membranes used were a gas-permeable silicone membrane, a hydrophobic polypropylene sample having a 0.04 μm effective pore sizes, and a PTFE membrane with 1.0 micron pore size selected because it was desirable to have maximum pore size that would unquestionably retain its hydrophobicity. The membranes included a silicone membrane having internal woven support fabric with a thickness of 160 μm (calipers), 180 μm (optical microscope). A Celegard membrane that is comprised of polypropylene, hydrophobic, laminated to 0.125 mm non-woven polypropylene and can have a thickness of about 25 μm and a pore size of about 0.04 μm. A GoreTex membrane comprised of PTFE, ydrophobic, laminated to polypropylene web with an effective pore size of about 1.0 μm and thickness between about 50 or 75 μm.

In some exemplary embodiments, the sorbents of the present disclosure can be any suitable composition to remove NH₃ from a solution of NH₄⁺. In initial testing of the composition, the sorbents can include citric acid dissolved in deionized or solids suspended in deionized water along with a ZP—H and Dowex.

Density and specific gravity can be measured in a graduated cylinder. First, the dry ion exchangers were put in the graduated cylinder and the weight and volume were measured. Then, deionized (DI) water was added to the cylinder to a measured volume. When the ion exchanger was thoroughly wetted, the weight and volume of the suspension was measured.

The weight of the same volume of DI water was also measured. With that information, the volume displaced by the sorbent can be calculated and knowing the weight of the sorbent allow calculation of the density relative to water (specific gravity). The initial condition of the Dowex was as it is supplied, which contains some water. Hydrogen-loaded zirconium phosphate can be supplied and have a pH when suspended in DI water or about 2.11. The powder can have a dry density of 1.11 g/mL with a specific gravity of about 2.45. The hydrogen exchange capacity of the ZP-H can be about 5.5 mEq/g with a particle size of about 40 μm aggregate. A Dowex cation exchange resin can be provided and include polystyrene sulfonate. The cation exchange resin can have a pH of about 2.39 when suspended in DI water with a density of about 0.67 g/ML. The specific gravity of Dowex can be about 1.18 with a hydrogen exchange capacity of about 2.8 mEq/g when hydrated. The particle size can be between about 50-100 mesh or about 150-300 μm. The citric acid can have a pH of 0.45 at 60% w/v and a pH of 2.08 at about 3% w/v. The test cells were filled with about 33 mL with a membrane area of about 14.5 cm² average.

The half-cells were loaded with the sorbent and NH₄Cl solution, respectively. NH₄Cl solution had added red food color, as a marker for membrane leaks. A small air bubble was left in each half-cell. Cells were incubated at room temperature. They were agitated on a rotating platform whose axis of rotation was parallel with the membrane, so that the bubble moved across the membrane and the back of the cell during each rotation.

Ammonium concentration in the small bowel is presumed to be approximately 14 mM. Test conditions for the sorption of ammonia by citric acid are shown in Table 1. The first experiments used extremely high ammonium and citric acid concentrations in order to accentuate the effect. When doing the GoreTex experiments, it was realized that a less extreme ammonium concentration would be more realistic and the amount of citric acid in 3% solution was still at least twice in excess of the amount of ammonium that would be transferred. The ion exchange sorbent experiment used 70 mM ammonium chloride solutions. All of the ammonium chloride solutions were buffered with 50 mM phosphate buffer to approx. pH 7.4 in order to approximate physiological conditions. Tests were conducted for at least 24 hours.

Experiments were done and reported in pairs. A third test cell was used during the last experiment, but one of the data sets had erratic results, which are not reported here.

TABLE 1
Test conditions for ammonia sorption.

| | [NH4Cl] | [Citric Acid] | [Citric Acid] |
|---|---|---|---|
| Membrane | mM | wt/vol | mM |
| Silicone | 140 | 60% | 3,123 |
| Celgard | 140 | 60% | 3,123 |
| Goretex | 70 | 3% | 156 |

| | | |
|---|---|---|
| Celgard | 70 | ZP or Dowex |

Samples of approx. 0.5 mL were taken during the experiment and the sorbent samples were filtered at about 0.2 microns. The solution pH was measured with a laboratory pH meter. Total nitrogen from NH₃ and NH₄⁺ in NH₄Cl solution and sorbent side was measured by colorimetric test kit B551 Urea Nitrogen produced by TECO Diagnostics. This kit uses a modified Berthelot reaction method for measurement of urea using the following equations:

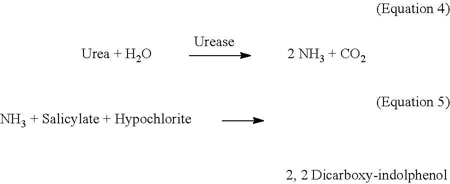

In the acidic conditions of the assay and the experiment, the NH₃ will be converted to NH₄⁺ and will thereby not evaporate quickly. The urease reaction is superfluous for our use of this reagent kit because all the nitrogen to be measured is already in the NH₃ or NH₄⁺ forms. This assay measures the total nitrogen from both NH₃ and NH₄⁺.

Concentration gradient was calculated two ways, based on either [total nitrogen] or [NH₃], as shown below. Assuming that the only species traversing the hydrophobic membrane is NH₃, the mass transfer coefficient based on [NH₃] would be more realistic. The amount and rate of nitrogen transfer are computed from the assayed total nitrogen. The total nitrogen concentration can be represented as [N] and can represent total ammoniacal nitrogen. For either the NH₄Cl or sorbent solution the nitrogen content (NC) can be found using the following equation:

$$NC = solution volume * [ N ] \qquad ( Equation 6 )$$

Nitrogen transfer (NT) can then be calculated during a time interval utilizing the following equation:

$$NT = Initial NC - NC at sample time \qquad ( Equation 7 )$$

Hydroxide concentration [OH⁻] can be calculated from the measured pH. Kₐ for water is 1.01*10⁻¹⁴, or pK_{w}=13.996 utilizing the Equation 8 below.

$$[ {OH}^{-} ] = {10}^{( pH - {pK}_{w} )} \qquad ( Equation 8 )$$

Ammonia equilibrium constant, K_{b} can be calculated using the equilibrium concentration equation gives us the following relationships. The only unknown in these relationships is the [NH₃]. The pKₐ for NH₃ is 9.25 and the following equation can be utilized.

$${K}_{b} = \frac{[ {NH}_{4}^{+} ] [ {OH}^{-} ]}{[ {NH}_{3} ]} = {10}^{( {pK}_{a} - {pK}_{w} )} = 1.796 * {10}^{- 5} \qquad ( Equation 9 )$$

Ammonia Concentration was calculated from the equilibrium constant and the pH. First, [OH] can be calculated from the measured pH. Kₐ for water is 1.01*10⁻¹⁴, or pk_{w}=13.996. Rearranging the first identity in Equation 8 to solve for [NH₃]. The [NH₄⁺] was assayed per above.

$$[ {NH}_{3} ] = \frac{[ {NH}_{4}^{+} ] [ {OH}^{-} ]}{{K}_{b}} \qquad ( Equation 10 A )$$

Since the measured nitrogen concentration is the total ammoniacal nitrogen, the measured nitrogen must be used for computing [NH₃].

$$[ {NH}_{4}^{+} ] = [ N ] - [ {NH}_{3} ] \qquad ( Equation 10 B )$$

Substituting Equation 10B into Equation 10A and solving for [NH₃],

$$[ {NH}_{3} ] = \frac{[ N ] [ {OH}^{-} ]}{{K}_{b} + [ {OH}^{-} ]} \qquad ( Equation 10 C )$$

For experiments where only the initial and final pH were measured, the intermediate pH values were linearly interpolated.

In this example, Equation 10D is used to calculate [NH₃]. Equation 10D results in an increase of approximately 1.0% in the calculated [NH₃] as compared to Equation 10C. This produces a decrease of approximately 1% in mass transfer coefficient. Compared to all the other experimental variabilities of this study, correcting this calculation is not significant at this time.

$$[ {NH}_{3} ] = \frac{[ N ] [ {OH}^{-} ]}{{K}_{b}} \qquad ( Equation 10 D )$$

The concentration gradient (CG) can equal the average concentration difference between NH₄Cl solution and sorbent solution. The CG can equal the average of both the (initial concentration difference, concentration difference at sample time). CG can be calculated for both Total Nitrogen and ammonia (NH₃) as shown below.

$${CG}_{( Total N )} = {[ N ]}_{NH 4 Cl solution} - {[ N ]}_{sorbent solution} \qquad ( Equation 11 A )$$

$${CG}_{( NH 3 )} = {[ {NH}_{3} ]}_{NH 4 Cl solution} - {[ {NH}_{3} ]}_{sorbent solution} \qquad ( Equation 11 B )$$

The mass transfer coefficient (MTC) equal the NT divided by the product of the CG, membrane area, and time interval as shown in the below equation.

$$MTC = NT / ( CG * area * time ) \qquad ( Equation 12 )$$

Two MTC can be calculated, one for total nitrogen and one for ammonia. These MTC can differ only by the concentration gradients used in the calculation. The total nitrogen MTC uses nitrogen assay data; the ammonia MTC uses ammonia concentrations calculated from nitrogen assay and pH measurement.

The MTC was not constant during the experiments, as shown below. Since the sampling was not done at the same time intervals during each experiment, it is desirable to compare MTC at the same sample times. To do this, a linear fit to the MTC data including the desired sample time was computed and the MTC was estimated at the standard time interval. This was done for time intervals of 3 and 24 hours. MTC does not include any properties of the membrane, such as thickness or porosity. The silicone membrane is a homogeneous phase and its transport phenomena will be different than the porous membranes polypropylene and PTFE membranes.

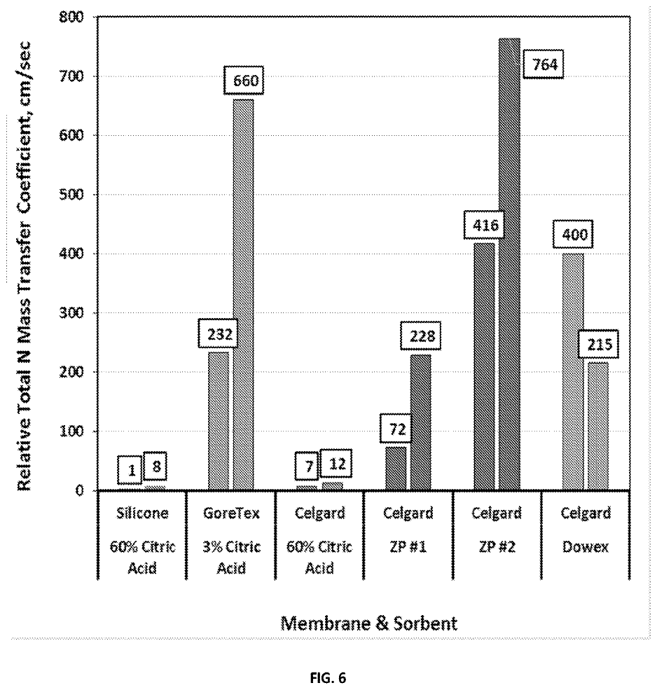

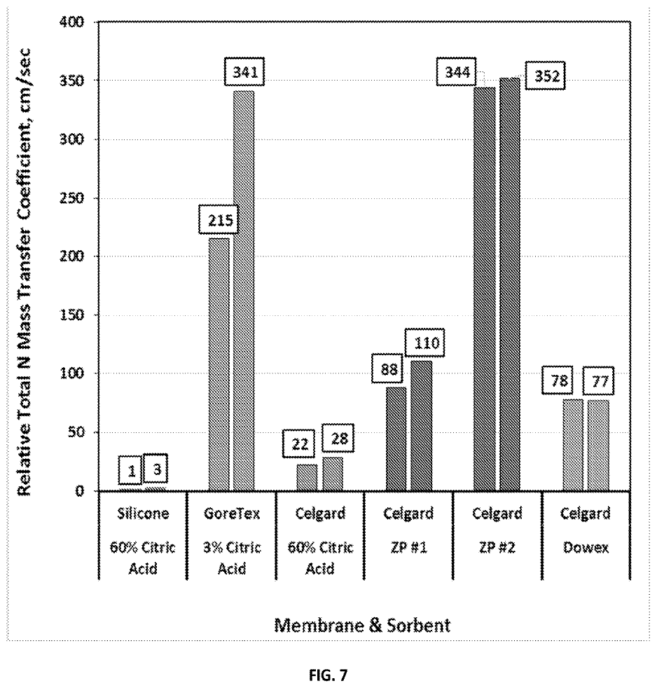

FIG. 6 and FIG. 7 show graphs illustrating the relative mass transfer coefficients at 3 and 24 hours, respectively, for the experiments herein reported. In each graph, all the coefficients are shown in proportion to the smallest coefficient of the data in that chart.

These mass transfer coefficients were calculated on the basis of the disappearance of total nitrogen from the NH₄Cl solution. The qualitative relationship between the various experiments is similar at the two time periods. At 24 hours, the MTC data in each pair are closer to each other and the magnitude difference between the smallest and largest MTC is about half the size, as compared to the data at 3 hours.

Experiments with citric acid as the sorbent were conducted with three membranes: silicone, GoreTex, and Celgard. The mass transfer performance of the silicone membrane with 60% citric acid was much less than any of the other experiments. The 3% citric acid with the GoreTex membrane had surprisingly large MTC. Compared to citric acid sorption with silicone and Celgard, it seems that the membrane is the major factor in the high MTC. GoreTex membrane seems to all transport of ammonia gas much more than Celgard.

Mass transport through Celgard to ZP was not consistent between the two experiments. At 3 hours, the Dowex sorbent performed much more like ZP than citric acid. At 24 hours, the ZP #1 performance is very similar to both the citric acid and Dowex, while the ZP #2 MTC is about 3.5 times the ZP #1.

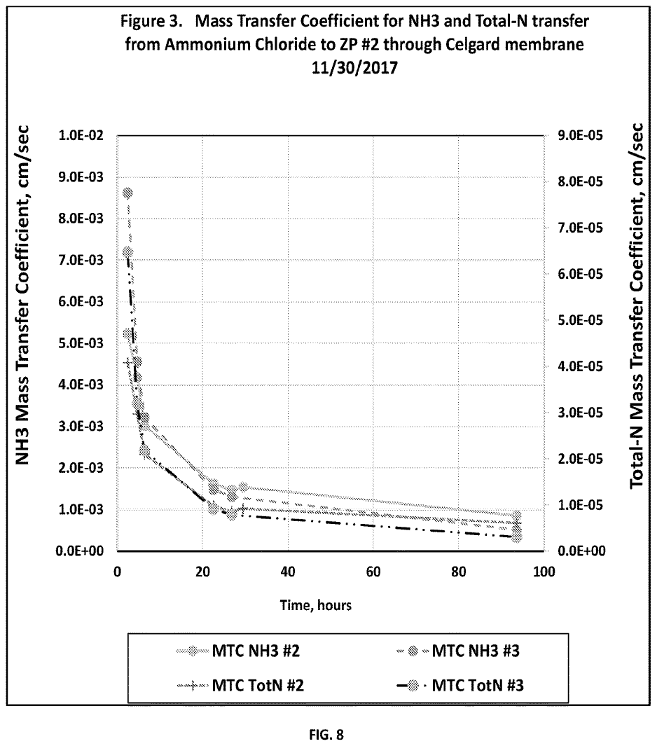

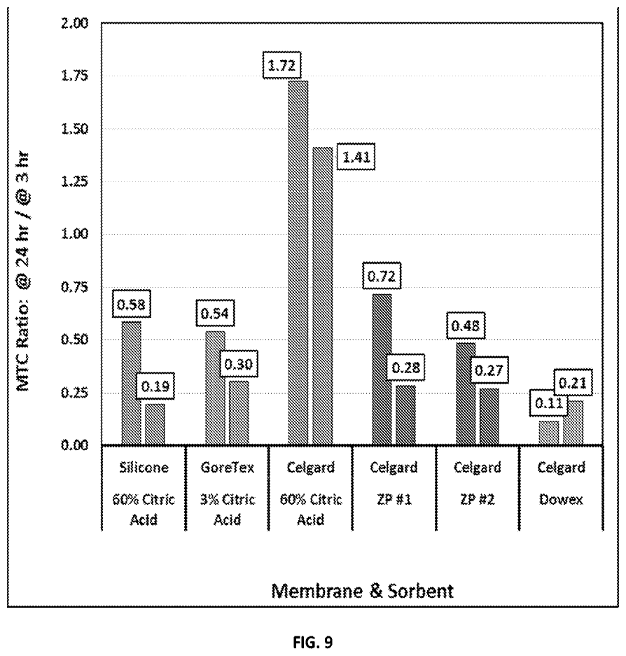

FIG. 8 and FIG. 9 show graphs illustrating the change of the mass transfer coefficient during an experiment. FIG. 8 shows the MTC for both total nitrogen and NH₃ for the second ZP experiment. As expected, the pattern of MTC over time is similar for both methods of calculating MTC, because they differ only by the concentration of the species of nitrogen that is moving. The NH3 MTC is larger by a factor of approximately 100 because its concentration is approximately 100-fold smaller.

FIG. 9 shows the change in MTC from 3 hours to 24 hour for each experiment. The MTC at 24 hours is divided by the 3-hour MTC for each test. In all cases except the Celgard membrane with 60% citric acid, the MTC significantly decreased.

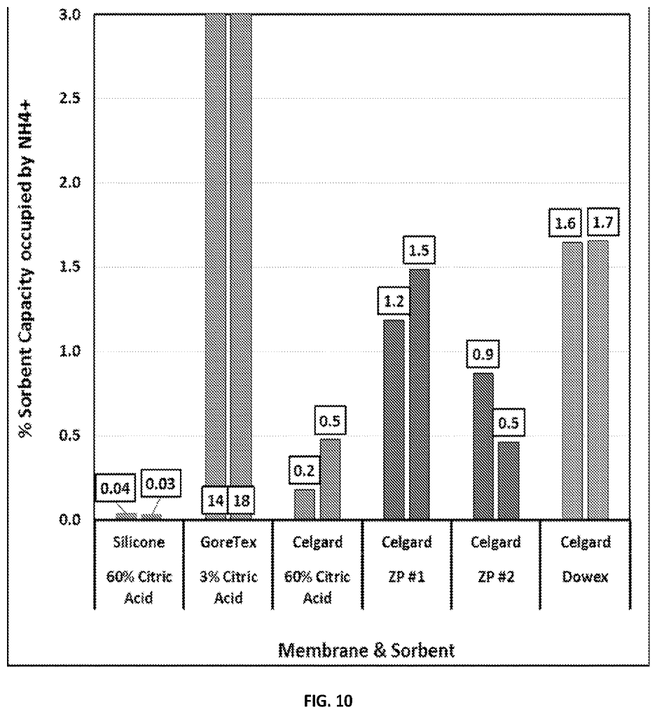

FIG. 10 illustrates the amount of total nitrogen adsorbed by the sorbent at 24 hours as a fraction of the total available hydrogen. The sorbent availability of hydrogen is much greater than the amount of ammonium that was adsorbed. The sorbent capacity was always much greater than the nitrogen transferred, so that the nitrogen in the sorbent should not have been a limiting factor in the transfer to sorbent.

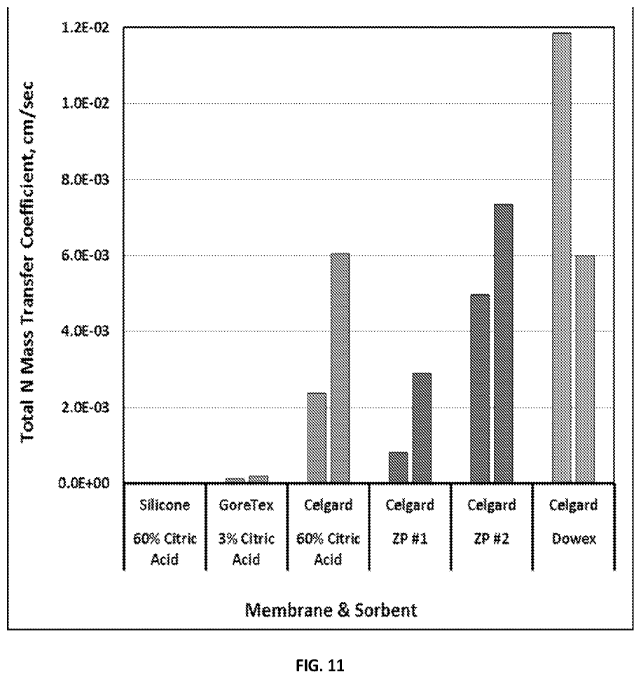

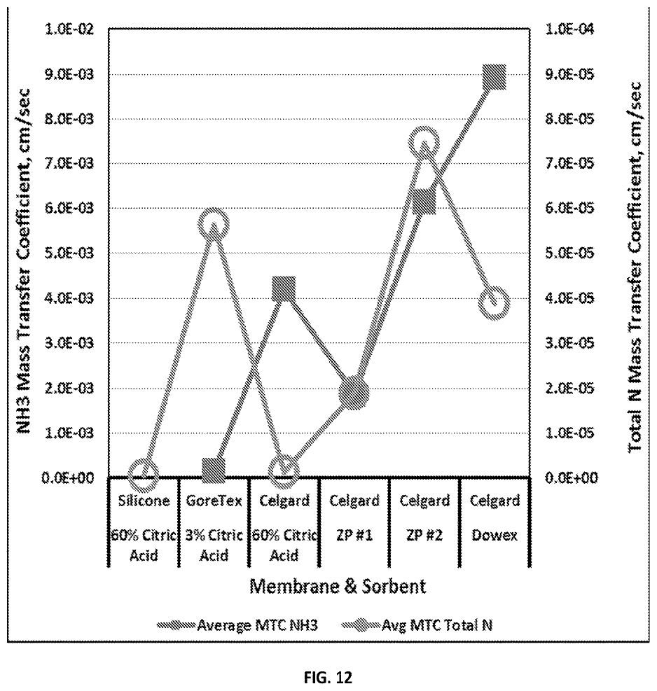

FIG. 11 and FIG. 12 illustrate the actual mass transfer coefficients. As expected, the MTC for ammonia is often much greater than when computed on the basis of total nitrogen.

The smaller concentration gradient produces a larger MTC. The citric acid experiments often were not as thoroughly sampled and analyzed as the experiments with ZP and Dowex. Consequently, the data necessary to generate the MTC were estimated or interpolated from the existing data.

The reason that the two sets of coefficients, for ZP and Dowex with Celgard membrane, are not proportional is that the concentration gradients are different. The pH of the sorbent drastically affects the ammonia concentration, from which the MTC is computed. In this embodiment, the ion exchangers performance in this scenario is better than citric acid. It will be much easier to encapsulate solid particles like Dowex or ZP than it would be to coat citric acid crystals. Based upon these experimental results, the ZP and Dowex may more effectively sequester the ammonia from the solution, providing a stronger driving force for ammonia to cross the hydrophobic membrane.

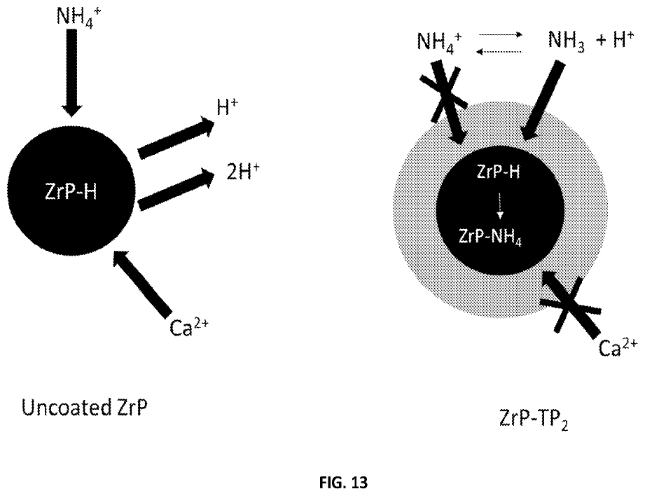

Accordingly, because ZP has the highest density of exchange capacity, is a versatile ion exchange material, and is also the least expensive, it appears to be an effective material for facilitating the removal of ammonium when surrounded by a gas permeable membrane. This is particularly beneficial as ZP is well tolerated when taken with food. FIG. 13 provides an exemplary embodiment of the composition of the present disclosure having a ZP coated with a gas permeable membrane.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. An oral sorbent composition, comprising:
a sorbent component; and a
gas permeable membrane coating, wherein the sorbent component is coated by the gas permeable membrane coating, wherein the gas permeable membrane coating allows for NH₃ or NH₄⁺ that would remove NH⁺ from a surrounding solution, wherein the NH₄⁺ is trapped inside the gas permeable membrane.

2. The oral sorbent composition of claim 1, wherein the sorbent component is an ion exchange material.

3. The oral sorbent composition of claim 2, wherein the gas permeable membrane coating is formed via bonding chemical bonding to the sorbent component.

4. The oral sorbent composition of claim 2, wherein the gas permeable membrane is configured to allow gas through and water vapor transfer, but no the passage of liquid water.

5. The oral sorbent composition of claim 4, wherein the sorbent component is comprised of hydrogen-loaded zirconium phosphate.

6. The oral sorbent composition of claim 2, wherein the gas permeable membrane coating is comprised of a perfluorocarbon composition.

7. A chemical composition comprising:
a gas permeable membrane;
a non-selective cation exchanger; and
wherein the gas permeable membrane surrounds the non-selective cation exchanger.

8. The composition of claim 7, wherein the non-selective cation exchanger is hydrogen-loaded zirconium phosphate (H—ZP).

9. The composition of claim 8, wherein the gas permeable membrane is comprised of a perfluorocarbon composition.

10. The composition of claim 9, wherein the perfluorocarbon composition is perfluorooctyltriethoxsilane.

11. The composition of claim 10, further comprising a coating layer applied to the non-selective cation exchanger and the non-selective anion exchanger, wherein the coating layer is also surrounded by the gas permeable membrane.

12. The composition of claim 11, wherein the coating layer is comprised of tetraethyl orthosilicate (TEOS).

13. The composition of claim 10, further comprising a non-selective anion exchanger, wherein the gas permeable membrane surrounds the non-selective anion exchanger.

14. The composition of claim 13, wherein the non-selective anion exchanger is hydroxide-loaded zirconium oxide (OH—ZO).

15. A method for removing ammonia from an aqueous environment, as a means to decrease urea level in the gut and body in treatment of kidney failure, the method comprising the steps of:
ingesting an oral sorbent having a gas permeable membrane.

16. The method of claim 3, wherein the oral sorbent comprises hydrogen-loaded zirconium phosphate or any similar hydrogen-loaded cation exchanger.
